(19) United States
(12) Patent Application Publication
(10) Pub. No.: US 2010/0256621 A1
(43) Pub. Date: Oct. 7, 2010

(54) SINGLE PHASE LIQUID REFRIGERANT CRYOABLATION SYSTEM WITH MULTITUBULAR DISTAL SECTION AND RELATED METHOD

(75) Inventors: Alexei BABKIN, Albuquerque, NM (US); Peter LITTRUP, Bloomfield Hills, MI (US); William NYDAM, Rancho Santa Fe, CA (US); Barron NYDAM, Rancho Santa Fe, CA (US)

Correspondence Address: Convergent Law Group LLP, P.O. BOX 1329, MOUNTAIN VIEW, CA, 94042, US

(73) Assignee: CRYOMEDIX LLC, San Diego, CA (US)

(21) Appl. No.: 12/754,457

(22) Filed: Apr. 5, 2010

Related U.S. Application Data
(60) Provisional application No. 61/167,057, filed on Apr. 6, 2009.

(51) Int. Cl. A61B 18/02

(52) U.S. Cl. 606/21; 606/25

(57) ABSTRACT

Single phase liquid refrigerant cryoablation systems and methods are described herein. The cryoablation systems drive liquid cryogen or refrigerant along a closed fluid pathway without evaporation of the liquid cryogen. A cryoprobe includes a distal energy delivery section to transfer energy to the tissue. A plurality of cooling microtubes positioned in a distal section of the cryoprobe transfer cryogenic energy to the tissue. The plurality of microtubes in the distal section are made of materials which exhibit flexibility at cryogenic temperature ranges, enabling the distal section of the cryoprobe to bend and conform to variously shaped target tissues.

### CROSS-REFERENCE TO RELATED APPLICATIONS

The present application claims the benefit of application Ser. No. 61/167,057, filed Apr. 6, 2009, entitled “Cryogenic System for Improved Cryoablation Treatment”.

### BACKGROUND OF THE INVENTION

This invention relates to cryoablation systems for treating biological tissues, and more particularly, to cryoablation probes using refrigerants in the liquid state and cryosurgical probes with multitubular distal ends.

Cryosurgical therapy involves application of extremely low temperature and complex cooling systems to suitably freeze the target biological tissues to be treated. Many of these systems use cryoprobes or catheters with a particular shape and size designed to contact a selected portion of the tissue without undesirably affecting any adjacent healthy tissue or organ. Extreme freezing is produced with some types of refrigerants that are introduced through the distal end of the cryoprobe. This part of the cryoprobe must be in direct thermal contact with the target biological tissue to be treated.

There are various known cryosurgical systems including for example liquid nitrogen and nitrous oxide type systems. Liquid nitrogen has a very desirable low temperature of approximately −200° C., but when it is introduced into the distal freezing zone of the cryoprobe which is in thermal contact with surrounding warm biological tissues, its temperature increases above the boiling temperature (−196° C.) and it evaporates and expands several hundred-fold in volume at atmospheric pressure and rapidly absorbs heat from the distal end of the cryoprobe. This enormous increase in volume results in a “vapor lock” effect when the internal space of the mini-needle of the cryoprobe gets “clogged” by the gaseous nitrogen. Additionally, in these systems the gaseous nitrogen is simply rejected directly to the atmosphere during use which produces a cloud of condensate upon exposure to the atmospheric moisture in the operating room and requires frequent refilling or replacement of the liquid nitrogen storage tank.

Nitrous oxide and argon systems typically achieve cooling by expansion of the pressurized gases through a Joule-Thomson expansion element such as a small orifice, throttle, or other type of flow constriction that are disposed at the end tip of the cryoprobe. For example, the typical nitrous oxide system pressurizes the gas to about 5 to 5.5 MPa to reach a temperature of no lower than about −85 to −65° C. at a pressure of about 0.1 MPa. For argon, the temperature of about −160° C. at the same pressure of 0.1 MPa is achieved with an initial pressure of about 21 MPa. The nitrous oxide cooling system is not able to achieve the temperature and cooling power provided by liquid nitrogen systems. Nitrous oxide and cooling systems have some advantages because the inlet of high pressure gas at room temperature, when it reaches the Joule-Thomson throttling component or other expansion device at the probe tip, precludes the need for thermal insulation of the system. However, because of the insufficiently low operating temperature, combined with relatively high initial pressure, cryosurgical applications are strictly limited. Additionally, the Joule-Thomson system typically uses a heat exchanger to cool the incoming high pressure gas using the outgoing expanded gas in order to achieve the necessary drop in temperature by expanding compressed gas. These heat exchanger systems are not compatible with the desired miniature size of probe tips that need to be less than 3 mm in diameter. Although an argon system is capable of achieving a desirable cryoablation temperature, argon systems do not provide sufficient cooling power and require very high gas pressures. These limitations are very undesirable.

Another cryoablation system uses a fluid at a near critical or supercritical state. Such cryoablation systems are described in U.S. Pat. Nos. 7,083,612 and 7,273,479. These systems have some advantages over previous systems. The benefits arise from the fluid having a gas-like viscosity. Having operating conditions near the critical point of nitrogen enables the system to avoid the undesirable vapor lock described above while still providing good heat capacity. Additionally, such cryosystems can use small channel probes.

However, challenges arise from use of a near-critical cryogen in a cryoablation system. In particular, there is still a significant density change in nitrogen once it is crossing its critical point (about 8 times)—resulting in the need for long pre-cooling times of the instrument. The heat capacity is high only close to the critical point and the system is very inefficient at higher temperatures requiring long pre-cooling times. Additionally, the system does not warm up (or thaw) the cryoprobe efficiently. Additionally, near-critical cryogen systems require a custom cryogenic pump which is more difficult to create.

Still other types of cryosystems are described in the patent literature. U.S. Pat. Nos. 5,957,963; 6,161,543; 6,241,722; 6,767,346; 6,936,045 and International Patent Application No. PCT/US2008/084004, filed Nov. 19, 2008, describe malleable and flexible cryoprobes. Examples of patents describing cryosurgical systems for supplying liquid nitrogen, nitrous oxide, argon, krypton, and other cryogens or different combinations thereof combined with Joule-Thomson effect include U.S. Pat. Nos. 5,520,682; 5,787,715; 5,956,958; 6074572; 6,530,234; and 6,981,382.

However, despite the above described systems, an improved cryoablation system using low pressure and cryogenic temperatures that is capable of excluding evaporation and “vapor lock” within a multitubular distal end of the cryoprobe is still desirable.

### SUMMARY OF THE INVENTION

A cryoablation system circulates liquid refrigerant along a flowpath. The flowpath is closed and the liquid refrigerant is not allowed to evaporate or otherwise change states along the flowpath. The cryoablation system includes a number of components along the flowpath. A container is provided which holds the liquid refrigerant at an initial pressure and initial temperature. In one embodiment the initial pressure is relatively low and the initial temperature is normal environmental temperature or room temperature. The system further includes a liquid pump operable to drive the liquid refrigerant along the flowpath and to increase the pressure of the liquid refrigerant to a predetermined pressure thereby forming a compressed liquid refrigerant. A cooling device or refrigerator cools the compressed liquid refrigerant to a predetermined cryogenic temperature which is lower than the initial temperature. The predetermined cryogenic temperature is equal to a temperature that is lethal to tissue. In another embodiment, the predetermined cryogenic temperature is less than or equal to −100 degrees Celsius, and in another embodiment the temperature is less than or equal to −140 degrees Celsius.

The system additionally includes a cryoprobe adapted to receive the compressed liquid refrigerant. The cryoprobe has various sections including an elongate shaft having a distal energy-delivery section and a distal tip. The distal energy delivery section includes a bundle of cooling microtubes and a bundle of return microtubes. The liquid refrigerant flows towards and away from said distal tip through the cooling and return microtubes respectively.

In one embodiment, the return microtubes are fluidly coupled to at least one cryogen return line which transports the liquid refrigerant to the container thereby completing a circulation flow path of the liquid refrigerant without the liquid refrigerant evaporating. A check valve or another pressure reducer can be positioned along the flowpath between the return line and the container to reduce the pressure of the liquid refrigerant prior to entering the container.

The distal end section may be rigid or shapeable. In a rigid embodiment, the microtubes are formed of a rigid material such as stainless steel.

In another embodiment, the distal end is shapeable, bendable, or flexible. The microtubes may be manufactured of a material that maintains flexibility in a full range of temperatures from −200° C. to ambient temperature of the environment such that the distal section remains flexible during operation.

The inventive shapeability may be adjusted and selected based on diameter, wall thickness, and material. In one embodiment, each of the microtubes has an inner diameter in a range between 0.05 mm and 2.0 mm, a wall thickness in a range of between about 0.01 mm and 0.3 mm, and or are formed of polyimide material.

In another embodiment, an insulated inlet line extends along the shaft of the cryoprobe and delivers the liquid refrigerant to the bundle or plurality of cooling microtubes. The cooling inlet line is heat insulated with an evacuated or vacuum space.

In another embodiment the system operates at relatively low pressure. The initial pressure is between 0.4 to 0.9 MPa and the compressed pressure along the flowpath after compression is between 0.6 to 1.0 MPa. This has an advantage of allowing operation with a small liquid pump.

In another embodiment the refrigerator of the cryoablation system includes a heat exchanger submerged in a liquid cryogen having the predetermined cryogenic temperature.

In another embodiment, the bundles of microtubes are sufficient to increase the surface area of cooling surfaces, and therefore increase the heat transfer (cooling) to the target tissue. The number of microtubes is in a range of 5 to 100 microtubes. The plurality of cooling microtubes may be positioned circumferentially about the bundle of return microtubes forming an annulus configuration.

In another embodiment a cryoprobe is adapted to circulate a compressed liquid refrigerant to and from its distal tip while maintaining the refrigerant in a liquid only state. The cryoprobe has various sections including an elongate shaft having a distal energy-delivery section and a distal tip. The distal energy delivery section includes a bundle of cooling microtubes and a bundle of return microtubes. The liquid refrigerant flows towards and away from said distal tip through the cooling and return microtubes respectively.

In another embodiment of the present invention the cryoablation system includes a second flowpath that warms the liquid refrigerant prior to entry into the cryoprobe. The cryoprobe delivers heat to the target tissue. A switch, valve or other means controls which flowpath is selected and consequently, whether heat or cyroenergy is applied through the active tubes of the cryoprobe to the tissue.

In another embodiment, a cryoablation method for applying cryoenergy to tissue includes moving a liquid refrigerant along an enclosed flowpath without the liquid refrigerant changing states. The method further includes positioning a distal section of the cryoprobe in the vicinity of the target tissue and transferring cryoenergy to the tissue through the walls of a plurality of cooling microtubes which extend along the distal section of the cryoprobe. The plurality of microtubes may be flexed such that the distal section conforms to the tissue targeted for ablation to increase transfer of energy to the tissue.

The microtubes in one embodiment extend annularly along the shaft and concentrically surround a set of inner return microtubes. The return microtubes return warmer liquid refrigerant to a proximal portion of the cryoprobe.

Another embodiment of the invention includes a cryoablation method for applying energy to a tissue having a curved surface wherein the method includes the step of driving a liquid refrigerant along a flowpath of a cryoablation system. The liquid refrigerant remains in a single state and does not reach its critical state as it moves along the flowpath.

The method further includes positioning a distal section of the cryoprobe in the vicinity of the target tissue and bending the distal section about the curved surface. The method further includes the step of forming an ice structure about the distal section wherein the ice structure is formed by applying cryoenergy through a plurality of cooling microtubes present in the distal section. The shape of the ice structure may take the form of an elongate member, a loop, a hook, or another shape selected by the operator.

Another embodiment of the invention is to use non-nitrogen refrigerants. Still another embodiment is to circulate the liquid refrigerant such that the conventional Joule-Thomson effect is excluded. Still another embodiment is to circulate the liquid refrigerant at a non-near critical state, such that the viscosity of the fluid is that of the fluid in its liquid state as the refrigerant moves along its flowpath. Still another embodiment is to circulate a refrigerant fluid wherein the fluid remains substantially incompressible as it moves along the flowpath.

The description, objects and advantages of the present invention will become apparent from the detailed description to follow, together with the accompanying drawings.

### BRIEF DESCRIPTION OF THE DRAWINGS

FIGS. 1A and 1B are phase diagrams corresponding to cooling and heating cycles of a liquid refrigerant used in a cryoablation system in accordance with the present invention.

FIG. 2 is a diagram of the boiling temperature of liquid nitrogen as a function of pressure.

FIG. 3 is a schematic representation of a cooling system for cryoablation treatment comprising a plurality of microtubes in the cryoprobe.

FIG. 4a is a cross sectional view of a distal section of a cryoprobe in accordance with the present invention.

FIG. 4b is an enlarged view of the distal tip shown in FIG. 4a.

FIG. 4c is an enlarged view of the transitional section of the cryoprobe shown in FIG. 4a.

FIG. 4d is an end view of the cryoprobe shown in FIG. 4a.

FIG. 4e is a cross sectional view taken along line 4e-4e illustrating a plurality of microtubes for transporting the liquid refrigerant to and from the distal tip of the cryoprobe.

FIGS. 5-7 show a closed loop, single phase, liquid refrigerant cryoablation system including a cryoprobe operating to generate various shapes of ice along its distal section.

FIG. 8 is a schematic representation of another cooling system for cryoablation treatment comprising a plurality of microtubes in the cryoprobe and a second flowpath for warming the liquid refrigerant.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

The invented cooling system for cryoablation treatment uses liquid refrigerants at low pressures and cryogenic temperatures to provide reliable cooling of the distal end of the cryoprobe and surrounding biological tissues to be ablated. The use of liquid refrigerants as the cooling means combined with a multitubular distal end of the cryoprobe eliminates refrigerant vaporization and significantly simplifies the cryosurgical procedure.

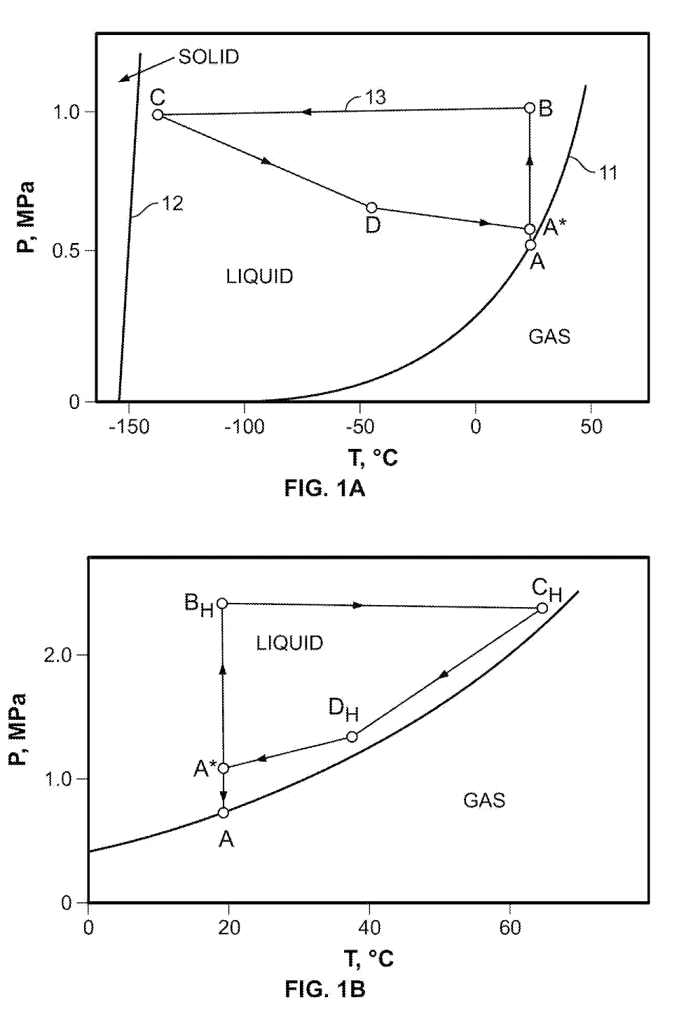

An example of the use of low pressure and cryogenic temperature refrigerants is illustrated in FIG. 1A. In particular, a phase diagram of R218 refrigerant (octafluoropropane) having a melting temperature of about −150° C. is shown. The axes of the diagram in FIG. 1A correspond to pressure p and temperature T of the R218 refrigerant, and include phase lines 11 and 12 that delineate the locus of points (p, T) where solid, liquid and gas states coexist. Although R218 is shown in connection with this embodiment, the invention may include use of other liquid refrigerants.

At point A of FIG. 1A, the refrigerant is in a “liquid-vapor” equilibrium state in a storage tank or container. It has a temperature T₀ of the environment, or slightly lower, at an initial pressure p₀ of about 0.4 MPa. The closed loop cycle or refrigerant flowpath begins at the point where the liquid refrigerant exits the container or storage tank. In order for the refrigerant to remain in the liquid state throughout the entire cooling cycle and provide necessary pressure for the cryogen to flow through a cryoprobe or a catheter it is maintained at a slightly elevated pressure in the range from about 0.7 to 0.8 MPa (or in this example about 0.75 MPa). This corresponds to point B of FIG. 1A. Point B is in the liquid area of R218 refrigerant. Further, the liquid is cooled by a cooling device (such as but not limited to a refrigerator) from point B to point C to a temperature Tₘᵢₙ that is shown by path 13 in FIG. 1A. This temperature will be somewhat higher (warmer) than its freezing temperature at elevated pressure.

The cold liquid refrigerant at point C is used for cryoablation treatment and directed into the distal end of the cryoprobe that is in thermal contact with the biological tissue to be treated. This thermal contact leads to a temperature increase of the liquid refrigerant with a simultaneous pressure drop from point C to point D caused by the hydraulic resistance (impedance) of the microchannel distal end of the cryoprobe. The temperature of the return liquid is increased due to its environment. In particular, the temperature is increased due to thermal communication with the ambient surroundings and by slightly elevated pressure maintained by a device, e.g., a check valve (point A*). A small pressure drop of about 6 kPa is desirable to maintain the liquid phase conditions in a return line that returns the liquid refrigerant back to the storage tank. Finally, the cycle or flowpath is completed at the point where the liquid cryogen enters the storage tank. Re-entry of the liquid refrigerant may be through a port or entry hole in the container corresponding once again to point A of FIG. 1A. The above described cooling cycle will be continuously repeated as desired.

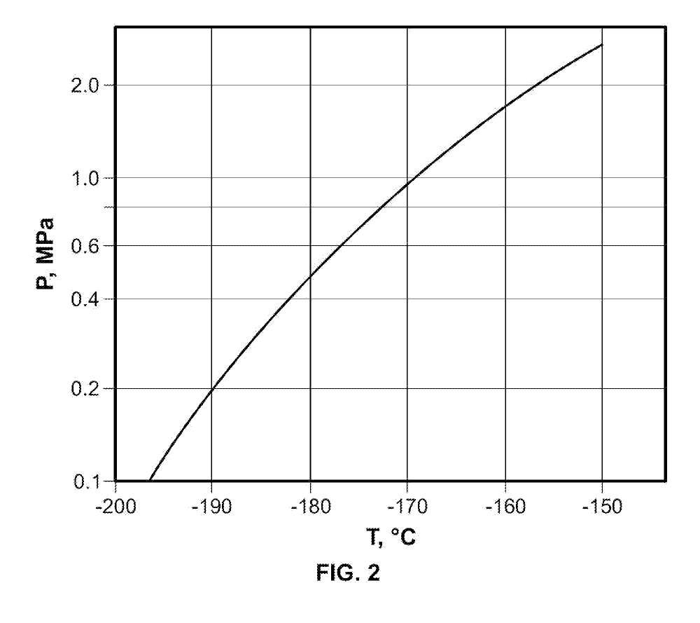

In some examples the cooling device or refrigerator can be a heat exchanger submerged in pressurized liquid nitrogen having a predetermined temperature Tₘᵢₙ depending on its pressure. The pressure may range from about 1.0 to 3.0 MPa. The liquid nitrogen can be replaced by liquid argon or krypton. In these cases, the predetermined temperatures Tₘᵢₙ will be obtained at pressures as low as about 0.1 to 0.7 MPa. An example of a “pressure, p—temperature, T” diagram of liquid nitrogen is shown in FIG. 2 defining the necessary predetermined temperature Tₘᵢₙ and corresponding pressure of the liquid refrigerant.

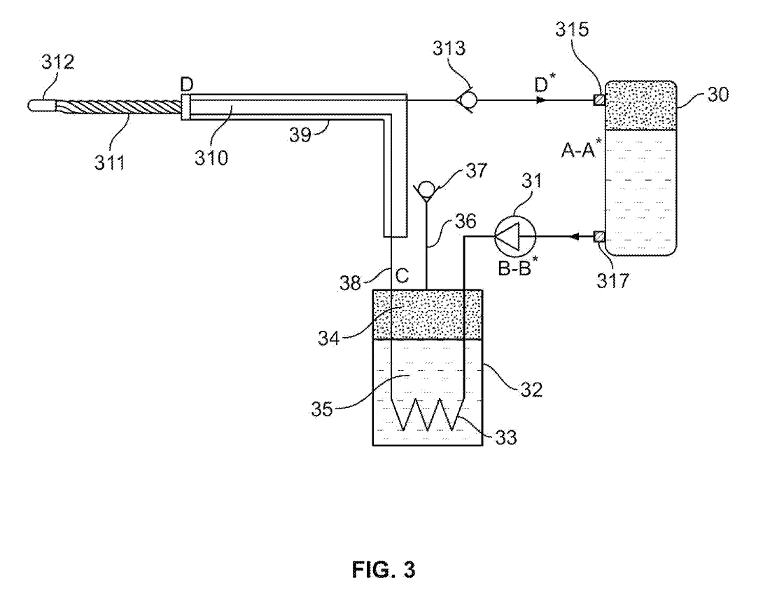

An embodiment of the invention is to circulate a refrigerant in its operational liquid state, in a closed loop, without any evaporation, under low pressure and low temperature during the cooling cycle. This cooling system for cryoablation treatment is schematically shown in FIG. 3 where the liquid refrigerant at initial pressure p₀ in container 30 is compressed by a liquid pump 31 under temperature T₀ of the environment. Contrary to typical closed cooling cycles where cooling is achieved by evaporating refrigerants followed by high compression of the vapor, this pump can be very small in size as it drives the incompressible liquid. Further, the liquid refrigerant is transferred into the refrigerator 32 through the coiled portion 33 which is submerged in the boil-off cryogen 34, 35 provided by transfer line 36 and maintained under a predetermined pressure by check valve 37.

The boil-off cryogen has a predetermined temperature Tₘᵢₙ. The coiled portion 33 of the refrigerator 32 is fluidly connected with multi-tubular inlet fluid transfer microtubes of the flexible distal end 311, so that the cold liquid refrigerant having the lowest operational temperature Tₘᵢₙ flows into the distal end 311 of the cryoprobe through cold input line 38 that is encapsulated by a vacuum shell 39 forming a vacuum space 310. The end cap 312 positioned at the ends of the fluid transfer microtubes provides fluid transfer from the inlet fluid transfer microtubes to the outlet fluid transfer microtubes containing the returned liquid refrigerant. The returned liquid refrigerant then passes through a check valve 313 intended to decrease the pressure of the returned refrigerant to slightly above the initial pressure p₀. Finally, the refrigerant re-enters the container 30 through a port or opening 315 completing the flowpath of the liquid refrigerant. The system provides continuous flow of a refrigerant, and the path A-B-C-D-A*-A in FIG. 3 corresponds to phase physical positions indicated in FIG. 1A. The refrigerant maintains its liquid state along the entire flowpath or cycle from the point it leaves the container through opening 317 to the point it returns to the storage tank or container via opening 315.

An example of a closed loop cryoprobe using a liquid refrigerant is described in U.S. patent application Ser. No. 12/425,938, filed Apr. 17, 2009, and entitled “Method and System for Cryoablation Treatment”.

In the present cooling system, the minimum achievable temperature Tₘᵢₙ of the described process is not to be lower than the freezing temperature of the liquid refrigerants to be used. For many practical applications in cryosurgery, the temperature of the distal end of the cryoprobe must be at least −100° C. or lower, and more preferably −140° C. or lower in order to perform a cryoablation procedure effectively. There are several commonly used non-toxic refrigerants that are known to have normal freezing temperatures at about −150° C. or lower as shown in the following TABLE 1.

TABLE 1

| | | Molecular | Normal | Normal |
|---|---|---|---|---|
| | Chemical | mass | freezing | boiling |
| Refrigerant | formula | (kg/mol) | point (° C.) | point (° C.) |
| R218 | C₃F₈ | 188.02 | −150 | −36.7 |
| R124 | C₂HClF₄ | 136.5 | −199 | −12.1 |
| R290 | C₃H₈ | 44.1 | −188 | −42 |
| R1270 | C₃H₆ | 42.08 | −185 | −47.7 |
| R600A | i-C₄H₁₀ | 58.12 | −159.5 | −11.8 |

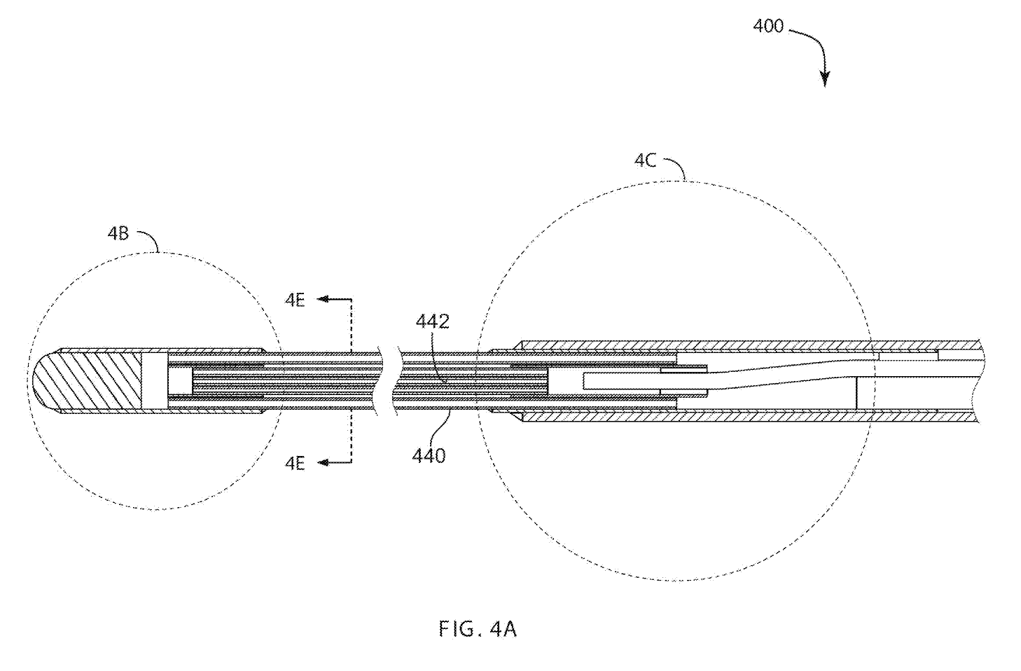

Referring to the FIG. 4a, a distal section 400 of a cryoprobe in accordance with one embodiment of the present invention is shown. The distal section 400 includes an energy-delivery section made up of a plurality of tubes 440, 442.

With reference to FIG. 4c and FIG. 4e, the distal section 400 includes two sets of tubes: inlet fluid transfer microtubes 440 and outlet fluid transfer microtubes 442. The inlet fluid transfer tubes 440 direct liquid refrigerant to the distal section of the cryoprobe creating a cryogenic energy delivering region to treat tissue in the vicinity of the probe. These cooling (or active) microtubes are shown in an annular formation. The outlet fluid transfer (or return) microtubes 442 direct liquid refrigerant away from the target site.

FIG. 4b is an enlarged view of the distal end of energy delivering section 400 shown in FIG. 4a. An end cap 443 is positioned at the ends of the inlet microtubes 440 and outlet microtubes 442, defining a fluid transition chamber 444. The transition chamber 444 provides a fluid tight connection between the inlet fluid transfer microtubes and the outlet fluid transfer microtubes. The end cap may be secured and fluidly sealed with an adhesive or glue. In one embodiment, a bushing 446 is used to attach plug 448 to the distal section. Other manufacturing techniques may be employed to make and interconnect the components and are still intended to be within the scope of the invention.

FIG. 4c illustrates an enlarged view of a transitional region 450 in which the plurality of cooling microtubes 440 are fluidly coupled to one or more larger inlet passageways 460 and the return microtubes are fluidly coupled to one or more larger return passageways 452. The return line(s) ultimately direct the liquid refrigerant back to the cryogen source or container such as, for example, container 30 described in FIG. 3 above, and thereby complete the flowpath or loop of the liquid cryogen and without allowing the cryogen to evaporate or escape.

In a preferred embodiment, the inlet line 460 is thermally insulated. Insulation may be carried out with coatings, and layers formed of insulating materials. A preferred insulating configuration comprises providing an evacuated space, namely, a vacuum layer, surrounding the inlet line.

The fluid transfer microtubes may be formed of various materials. Suitable materials for rigid microtubes include annealed stainless steel. Suitable materials for flexible microtubes include but are not limited to polyimide (Kapton). Flexible, as used herein, is intended to refer to the ability of the multi-tubular distal end of the cryoprobe to be bent in the orientation desired by the user without applying excess force and without fracturing or resulting in significant performance degradation. This serves to manipulate the distal section of the cryoprobe about a curved tissue structure.

In another embodiment flexible microtubes are formed of a material that maintains flexibility in a full range of temperatures from −200° C. to ambient temperature. In another embodiment materials are selected that maintain flexibility in a range of temperature from −200° C. to 100° C.

The dimensions of the fluid transfer microtubes may vary. Each of the fluid transfer microtubes preferably has an inner diameter in a range of between about 0.05 mm and 2.0 mm and more preferably between about 0.1 mm and 1 mm, and most preferably between about 0.2 mm and 0.5 mm. Each fluid transfer microtube preferably has a wall thickness in a range of between about 0.01 mm and 0.3 mm and more preferably between about 0.02 mm and 0.1 mm.

The present invention provides a substantial increase in the heat exchange area over previous probes. The heat exchange area of the present invention is relatively larger because of the multi-tubular nature of the distal end. Depending on the number of microtubes used, the distal end can increase the thermal contact area several times over previous distal ends having similarly sized diameters with single shafts. The number of microtubes may vary widely. Preferably the number of microtubes in the shaft distal section is between 5 and 100, and more preferably between 20 and 50.

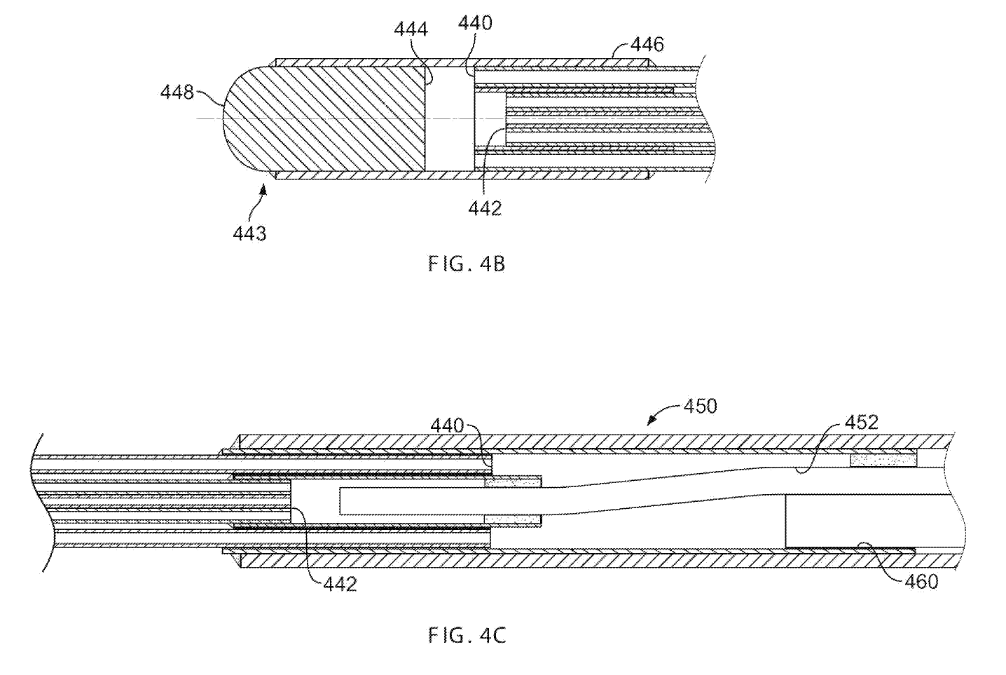

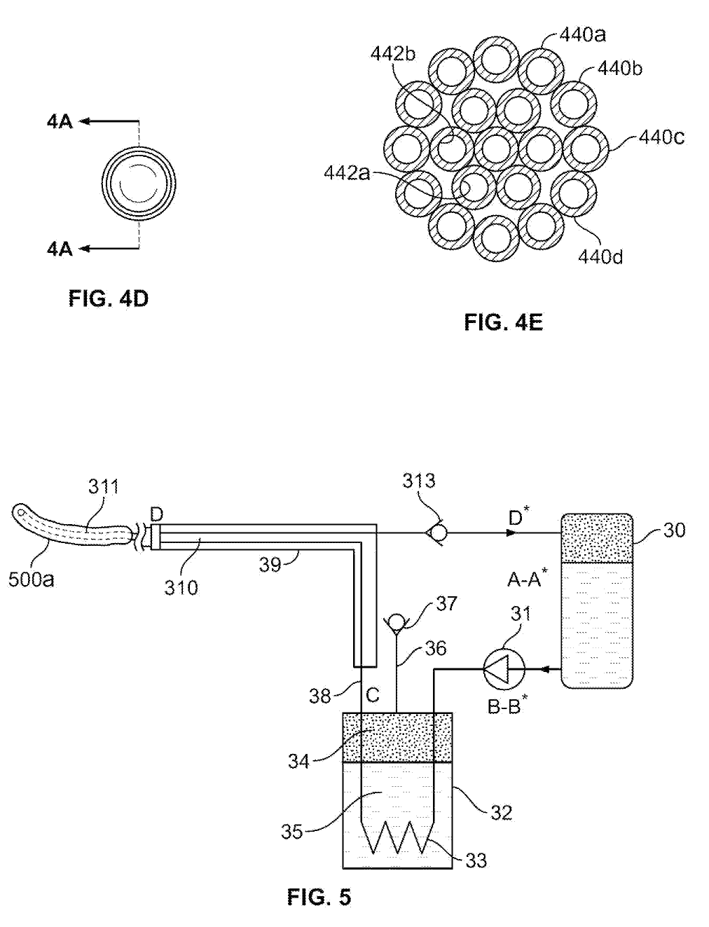

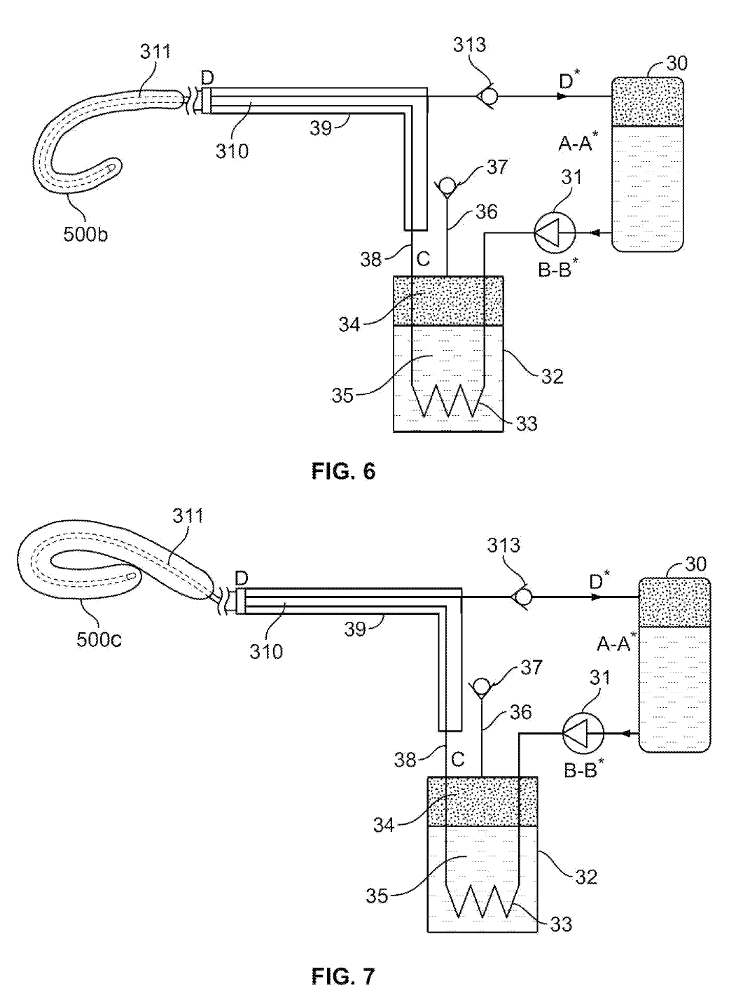

As can be seen in FIGS. 5-7, different shapes of ice structures and iceballs 500a, b, c, may be generated about the multi-tubular distal section 311 of the cryoprobe. It can be seen that an iceball can be created in a desired shape by bending the distal end in the desired orientation. These shapes may vary widely and include, e.g., an elongate member 500a of FIG. 5, a hook 500b of FIG. 6, a complete loop 500c as shown in FIG. 7, or an even tighter spiral (“fiddlehead fern”). See also, International Patent Application No. PCT/US2008/084004, filed Nov. 19, 2008, for another type of multitubular cryoprobe.

Another embodiment of the present invention includes heating the distal section of the cryoprobe. Warming the distal section of the cryoprobe may serve to thaw an ice structure, to facilitate probe removal, or to provide a surgical application such as but not limited to electrocautery, coagulation or heat based ablation.

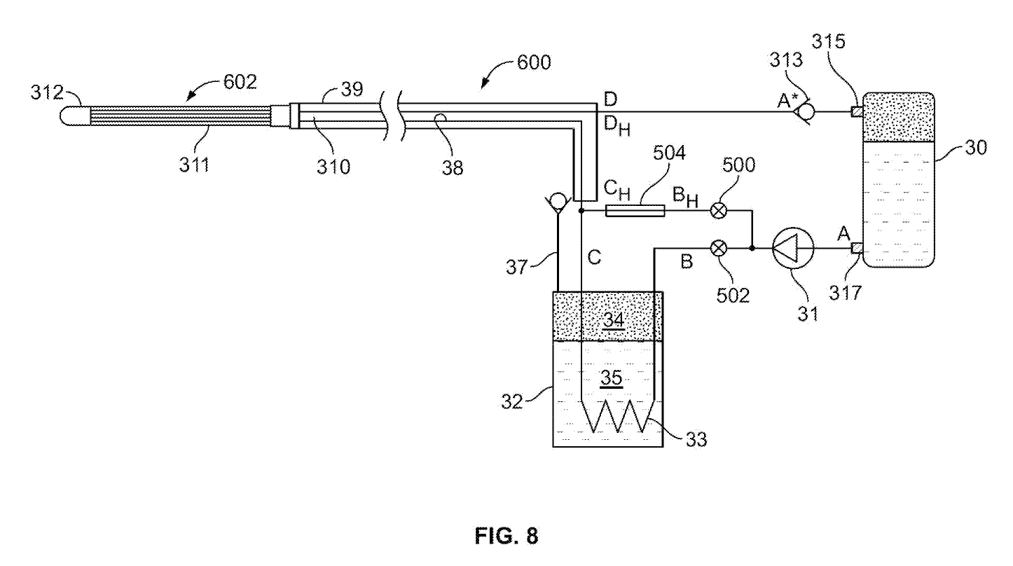

FIG. 8 shows a cryoablation system including a first cooling flowpath ABCDA*as described above in connection with FIGS. 1A and 3 and a second warming flowpath AB_{H}C_{H}D_{H}A* for warming the liquid. In particular, the warming flowpath commences at storage tank 30 of FIG. 8 and corresponds to Point A* of FIG. 1B. The liquid refrigerant is compressed by liquid pump 31 corresponding to the point B_{H} of FIG. 1B.

As shown in FIG. 8, the liquid refrigerant bypasses the refrigerator 32 and enters a heating unit 504. Bypassing the refrigerator, or switching the flowpaths may be performed using, for example, valves 500, 502. However, other means may be utilized as is known to those of skill in the art.

The heater 504 may be an inline heater which raises the temperature of the liquid, and corresponds to point CH of FIG. 1B.

The liquid exits that heater section and enters the cryoprobe or catheter 600. The warmer liquid thermally communicates with tissue/ice via the distal section 602 and the multitubular structure.

The liquid refrigerant exits the catheter and assumes a temperature and pressure corresponding to that shown at point DH of FIG. 1B. The liquid next assumes the environmental temperature at the point A* after which is returned back to the storage tank via port 315. Check valve or another means 313 may be incorporated to provide a small pressure difference between A* and A that maintains the cryogen in its liquid state throughout the entire flowpath and cycle.

The capability of the multi-tubular distal end of the cryoprobe extends cryoablation from a rigid needle-like application to nearly any current device used to assist current diagnostic and therapeutic procedures including but not limited to external and internal cardiac applications, endoscopic applications, surgical tools, endovascular uses, subcutaneous and superficial dermatologic applications, radiological applications, and others.

It will be understood that some variations and modification can be made thereto without departure from the spirit and scope of the present invention.

We claim:

1. A closed loop, single phase, liquid refrigerant cryoablation system for treating tissue comprising:
a container holding the liquid refrigerant at an initial pressure and initial temperature;
a liquid pump operable to increase the pressure of said liquid refrigerant to a predetermined pressure thereby forming a compressed liquid refrigerant;
a cooling device operable to cool the compressed liquid refrigerant to a predetermined cryogenic temperature, said predetermined cryogenic temperature lower than said initial temperature; and
a cryoprobe coupled to said cooling device and adapted to receive said compressed liquid refrigerant, said cryoprobe further comprising an elongate shaft having a distal energy-delivery section and distal tip, said energy delivery section comprising a plurality of cooling microtubes and a plurality of return microtubes wherein said liquid refrigerant flows towards and away from said distal tip through said cooling and return microtubes respectively and wherein said plurality of return microtubes are fluidly coupled to said container thereby completing the loop of said liquid refrigerant without said liquid refrigerant evaporating as the refrigerant is transported along the loop.

2. The system of claim 1 wherein said plurality of cooling microtubes circumferentially surround said plurality of return microtubes.

3. The system of claim 1 wherein said plurality of cooling microtubes and said plurality of return microtubes form a twisted bundle.

4. The system of claim 1 wherein each of said microtubes is manufactured of a material that maintains flexibility in a range of temperatures from −200° C. to ambient temperature of the environment such that said distal section remains flexible during operation.

5. The system of claim 1 wherein said cooling microtubes are connected to a cooling input line, and said input line being insulated by a vacuum space.

6. The system of claim 1 wherein said predetermined cryogenic temperature is less than or equal to −140° C.

7. The system of claim 1 wherein said initial pressure is between 0.2 to 1.5 MPa and said predetermined pressure is between 0.6 to 2.0 MPa.

8. The system of claim 6 wherein said cooling device is a refrigerator and comprises a coiled heat exchanger submerged in a liquid cryogen having said predetermined cryogenic temperature.

9. The system of claim 6 wherein said cooling device is one selected from a Stirling and a pulse tube cryocooler.

10. The system of claim 1 wherein each of said microtubes has an inner diameter in a range between 0.1 mm and 1.0 mm.

11. The system of claim 1 wherein each of said microtubes has a wall thickness in a range of between about 0.01 mm and 0.3 mm.

12. The system of claim 1 wherein each of said microtubes is formed of polyimide material.

13. The system of claim 1 wherein said liquid refrigerant is R218.

14. A single phase liquid refrigerant cryoablation system for treating tissue comprising:
a liquid refrigerant;
a container holding the liquid refrigerant at an initial pressure and initial temperature, the container comprising an entrance and an exit for the liquid refrigerant to enter and exit respectively, said entrance defining the beginning of a liquid refrigerant flowpath and said exit defining the end of said refrigerant flowpath;
a liquid pump in fluid communication with said container and operable to drive said liquid refrigerant from said container along the flowpath and to increase the pressure of said liquid refrigerant to a predetermined pressure thereby forming a compressed liquid refrigerant;
a cooling device disposed along said flowpath and downstream of said pump and operable to cool the compressed liquid refrigerant to a predetermined cryogenic temperature, said predetermined cryogenic temperature lower than said initial temperature; and
a cryoprobe disposed along said flowpath and downstream of said refrigerator, said cryoprobe further comprising an elongate shaft having a distal energy-delivery section, said energy delivery section comprising a plurality of active microtubes for transporting said liquid refrigerant towards said tissue and a plurality of return microtubes for transporting said liquid refrigerant away from said tissue and wherein the liquid refrigerant remains in a liquid-only state along the flowpath.

15. The system of claim 14 further comprising a controllable cooling bypass loop, said bypass loop comprising a warming line which directs the liquid refrigerant away from the cooling device and causes the temperature of said liquid refrigerant to increase above that of ambient temperature prior to entering the cryoprobe.

16. A cryoablation method for applying cryoenergy to tissue comprising the steps of:
driving a liquid refrigerant along a first flowpath commencing at an outlet of a refrigerant container, through a cryoprobe having an energy delivering distal section, and back to an inlet of said refrigerant container wherein said liquid refrigerant remains in a liquid-only state along the first flowpath;
positioning said distal section of said cryoprobe in the vicinity of said tissue;
transferring cryoenergy to said tissue through the walls of a plurality of microtubes extending along said distal section of said cryoprobe.

17. The method of claim 16 further comprising conforming said distal section of said cryoprobe to said tissue to increase transfer of energy to said tissue wherein said conforming step is carried out by flexing the plurality of microtubes.

18. The method of claim 16 wherein said plurality of microtubes extend in an annular formation of said distal section.

19. The method of claim 16 wherein the positioning step is carried out through one device selected from the group consisting of an endoscope, a visualization device and a steering device.

20. The method of claim 16 further comprising the step of transferring heat to said tissue through the walls of the microtubes.

21. The method of claim 20 comprising switching the liquid refrigerant from said first flowpath to a second flowpath wherein said second flowpath includes a heating element that serves to warm the liquid refrigerant.

22. A cryoablation method for applying energy to a tissue having a curved surface, said method comprising:
driving a liquid refrigerant along a closed first flowpath of a cryoablation system without said liquid refrigerant changing states, said cryoablation system comprising a cryoprobe having a distal section;
positioning said distal section of said cryoprobe in the vicinity of said tissue;
bending said distal section;
forming an ice structure about said distal section and in contact with said tissue wherein said ice structure is formed by applying cryoenergy through a plurality of microtubes in said distal section.

23. The method of claim 22 wherein the shape of the ice structure is one shape selected from the group consisting of a loop, a hook, and a fiddlehead fern.

24. The method of claim 22 further comprising the step of melting said ice structure by applying heat energy to the ice through the walls of the microtubes.

25. The method of claim 23 comprising switching the liquid refrigerant from said first flowpath to a second flowpath wherein said second flowpath includes a heating element that serves to warm the liquid refrigerant.

26. The system of claim 1 wherein said liquid refrigerant is propane.
